(19) United States
(12) Patent Application Publication
(10) Pub. No.: US 2004/0053912 A1
(43) Pub. Date: Mar. 18, 2004

(54) Process for the preparation of a beta -lactam nucleus and the application thereof

(76) Inventors: Thomas van der Does, Garrucha (ES); Rienk Henrik Kuipers, Gouda (NL); Marjon Brigitte Diender, Ellicott City, MD (US); Jeroen Leonardus den Hollander, Oslo (NO); Adrianus Johannes Jozef Straathof, Delft (NL); Lucas Antonius Maria van der Wielen, Bleiswijk (NL); Joseph Johannes Heijnen, Rijen (NL)

Correspondence Address: Pillsbury Winthrop, Intellectual Property Group, 1600 Tysons Boulevard, McLean, VA, 22102, US

(21) Appl. No.: 10/344,639

(22) PCT Filed: Aug. 27, 2001
(86) PCT No.: PCT/NL01/00626

(30) Foreign Application Priority Data
Aug. 28, 2000 (NL) 1016032

(51) Int. Cl. A61K 31/43; C12P 37/06

(52) U.S. Cl. 514/192; 435/44

(57) ABSTRACT

The invention relates to a process for the preparation of an aqueous solution or suspension of a β-lactam nucleus and application thereof. The aqueous solution or suspension of the β-lactam nucleus is prepared by process wherein an enzymatic deacylation of a β-lactam compound, which compounds comprises a β-lactam nucleus with a side chain coupled to it via an amide bond and which deacylation reaction is carried out in a mixture of water and an organic solvent and which deacylation leads to a β-lactam nucleus and a carboxylic acid, is carried out at a pH value of between 2 and 6 so that the carboxylic acid is extracted in situ to the organic solvent.

The invention relates to a process for the preparation of an aqueous solution or suspension of a β-lactam nucleus, to an aqueous solution or suspension of a β-lactam nucleus obtainable with the process and to a process for the enzymatic preparation of a β-lactam antibiotic using the solution or suspension.

EP 0 826 776 discloses a process wherein a solution containing 6-aminopenicillanic acid (6-APA) is produced, which 6-APA solution may be employed directly, that is, without a working-up step, in a synthesis reaction. The process of EP 0 826 776 contains to that end a first step in which a fermentation broth comprising penicillin G is purified through ultrafiltration. In a second step the penicillin in the filtrate is enzymatically converted into a solution that contains 6-APA, phenylacetic acid and inorganic salts. The enzymatic conversion is carried out in such a way that the penicillin is rapidly converted. In a third step the enzymatic conversion products are separated across a series of resin columns. That process results in a 6-APA solution that may be used directly in the enzymatic preparation of penicillins of the ampicillin-or amoxicillin-type, or from which 6-APA may be crystallized. In addition a phenyladetic acid solution and a solid waste stream is formed.

The drawback of the process described in EP 0 826 776 is that this process is highly laborious.

The process according to the invention provides an alternative to the described route.

To that end, the process is characterized in that an enzymaric deacylation of a β-lactam compound, which compound comprises a β-lactam nucleus with a side chain coupled to it via an amide bond, and which deacylation is carried out in a mixture of water and an organic solvent and which deacylation leads to a β-lactam nucleus and a carboxylic acid, is carried out at a pH value of between 2 and 6. By using a mixture of water and an organic solvent the carboxylic acid is extracted in situ to the organic solvent. The amount of carboxylic acid that is extracted to the organic solvent may vary and is for example 60% of the amount of carboxylic acid formed during the deacylation reaction. Preferably, at least 70%, more preferably at least 90% and still more preferably at least 95% of the carboxylic acid is extracted to the organic solvent.

Most preferably, the carboxylic acid is extracted almost completely, still more preferably the carboxylic acid is extracted completely to the organic solvent.

An advantage of the process according to the invention is that it may lead to an aqueous solution or suspension of the β-lactam nucleus that does not contain any inorganic salts.

Preferably the enzymatic deacylation in the process according to the invention is carried out at a pH value greater than or equal to 3.5. The pH value preferably is lower than or equal to 5.

In the context of the present application, enzymatic deacylation is understood to be a reaction wherein the side chain is enzymatically removed from a β-lactam compound consisting of a β-lactam nucleus with a side chain coupled to it via an amide bond. The side chain is liberated in the form of a carboxylic acid.

Enzymatic deacylation reactions are known to those skilled in the art. In principle, any enzyme capable of cleaving the side chain of the β-lactam compound may be applied in the process according to the invention. Enzymes suitable for deacylation reactions are for example known as penicillin acylases or penicillin amidases. These enzymes are classified as E.C. 3.5.1.11. Such enzymes may be isolated from for example micro-organisms such as fungi and bacteria. Organisms known to produce penicillin acylases are for example Acetobacter, Aeromonas, Alcaligenes, Aphanocladium, Bacillus sp, Cephalosporius, Escherichia, Flavobacterium, Kluyvera, Mycoplana, Protaminobacter, Pseudomonas and Xanthomonas.

Also suitable is for example an enzyme capable of converting 7-β-4-carboxybutanamido-cephalosporanic acid into 7-aminocephalosporanic acid and glutaric acid. This enzyme may be recovered from Pseudomonas sp.

Preferably the enzyme is used in immobilised form.

Suitable β-lactam compounds to be applied in the deacylation reaction are for example β-lactam compounds according to Formula (I):

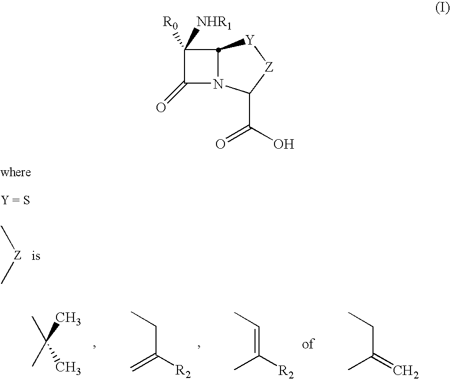

Ris —H;

R₁ is a side chain;

R₂ is —H. —H₃, —Cl, —CH=CH₂, —CH═C(CH₃)H.

As is known in the art, the structure shown in Formula (I) is typical for a β-lactam nucleus. The meaning of the variables, however, is not limited to those given in Formula (I).

Preferably, the starting material for the deacylation reaction is a β-lactam compound wherein R₁ is

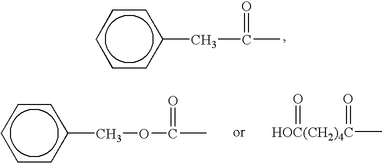

If a β-lactam compound according to Formula (I) is used in the deacylation reaction, a carboxylic acid according to Formula (II) is formed:

R₁—OH (II)

Suitable β-lactam compounds that may be employed in the process according to the invention are for example penicillin G (Pen G), 7-phenylacetamido-desacetoxy-cephalosporanic acid and N-adipoyl-7-aminodesacetoxycephalosporanic acid. These compounds are generally produced by fermentation processes. Preferably penicillin G is used as β-lactam compound. When Pen G is used in the process according to the invention, aqueous solutions or suspensions of 6-APA are obtained. If 7-phenylacetamido-desacetoxy-cephalosporanic acid or N-adipoyl-7-aminodesacetoxycephalosporanic acid is used, aqueous solutions or suspensions of 7-ADCA are obtained.

Various organic solvents may be applied in the process according to the invention. However, after mixing it with water, the organic solvent and water must be able to form two phases that can be separated. A suitable organic solvent is an organic solvent to which the carboxylic acid exhibits greater affinity than the β-lactam compound exhibits for the same organic solvent. A suitable solvent may readily be identified by carrying out the deacylation reaction at a pH in the region of the pKa of the carboxylic acid. A good solvent is a solvent that results in a conversion of the β-lactam compound in a β-lactam nucleus and a carboxylic acid which conversion in the presence of the organic solvent is higher than the conversion of the β-lactam compound in water, compared at equal pH values.

Distribution of the β-lactam compound is defined as the concentration of β-lactam compound in the organic solvent divided by the concentration of β-lactam compound in the aqueous phase. Distribution of the carboxylic acid is defined as the concentration of carboxylic acid in the organic phase divided by the concentration of carboxylic acid in the aqueous phase. An organic solvent is preferably a solvent that produces a low distribution of the β-lactam compound at a pH in the region of the carboxylic acid's pKa and a high distribution of the carboxylic acid, so that during the enzymatic deacylation the β-lactam compound and the carboxylic acid are so distributed among the aqueous phase and the organic phase that the aqueous phase becomes rich in the β-lactam compound and lean in carboxylic acid, and the organic phase becomes lean in β-lactam compound and rich in carboxylic acid. In other words, preferred solvents result in a concentration of β-lactam nucleus in the organic phase that is smaller than the concentration of carboxylic acid in the organic phase, while the concentration of the β-lactam nucleus in the aqueous phase is higher than the concentration of carboxylic acid in the aqueous phase.

A pH in the region of the pKa is for example a pH having a value between pKa−2 and pKa+2. Preferably, in identifying a suitable solvent, the pH has a value equal to the value of pKa plus or minus one.

Most preferably, an organic solvent is used that is favourable for a high yield in the deacylation reaction and for extracting the β-lactam compound from an aqueous solution to the organic solvent. Solvents that are apolar are not suitable to be applied in the process according to the invention. Suitable solvents are for example acetates, alcohols, ketones, esters and ethers. Preferred are C₁-C₅ acetates, C₁-C₆ alcohols. Preferably, solvents are used that are usual solvents to be applied on an industrial scale. Preferably, non-halogenated solvents are used. Highly suitable solvents for the process according to the invention wherein the β-lactam compound is penicillin G are for example n-butyl acetate and methyl-t-butyl ether (MTBE).

A solution of a free acid of a β-lactam compound in an organic solvent may be obtained by dissolving a free acid of a β-lactam compound in an organic solvent or by dissolving a salt of the β-lactam compound in water, followed by acidification and extraction to the solvent or by extraction of acidified fermentation broth or acidified fermentation broth filtrate that contains the β-lactam compound.

In a preferred embodiment the process is carried out by contacting a solution that contains a free acid of a β-lactam compound in an organic solvent with a solution or suspension of an enzyme in water, whereby the β-lactam compound is enzymatically converted into a β-lactam nucleus and a carboxylic acid at a pH value in the range from 2 to 6 and whereby an organic phase rich in carboxylic acid and an aqueous phase rich in the β-lactam nucleus are formed, which phases may be recovered separately. Separating aqueous and organic layers is known to those skilled in the art.

The aqueous phase which is rich in β-lactam nucleus and lean in carboxylic acid may be applied directly in a subsequent reaction.

Preferably the subsequent reaction is an enzymatic coupling reaction in which a side-chain precursor is coupled to a β-lactam nucleus with the aid of an enzyme. Enzymatic coupling reactions are known and are described in for example WO92/01601 and WO99/20786

It is known that the presence of the carboxylic acid is a problem in enzymatic coupling reactions because the presence of the carboxylic acid usually has an adverse effect on the activity or selectivity of the known enzymes for the coupling reaction. This is described for example in European patent application EP-A-0734452. In general the negative influence of the carboxylic acid is greater when the acid is present in larger quantities. Therefore, reaction mixtures obtained by an enzymatic deacylation reaction are not in general suitable for use in subsequent enzymatic reactions unless they are subjected to a purification step

The invention also relates to an aqueous solution or suspension of a β-lactam nucleus obtainable by the process according to the invention.

Such aqueous solutions and suspensions are highly suitable for direct application in a process for the enzymatic preparation of β-lactam antibiotics.

β-lactam antibiotics are known to those skilled in the art and are described in for example Kirk-Othmer ‘Encyclopeadia of Chemical Technology (3^{rd} edition, Volume 2, pages 871-915, John Wiley & Sons, New York). Preferably, a β-lactam antibiotic is a compound according to Formula I, wherein the side chain R₁ is not the same as R₁ present in the β-lactam compound used in the deacylation reaction.

An advantage of the process according to the invention is that the loss of β-lactam nucleus in comparison with the existing processes may be strongly reduced, because the β-lactam nucleus need not be isolated from the aqueous phase. The known processes involve a substantial loss of β-lactam nucleus, because, after isolation of the nucleus, a substantial amount of β-lactam nucleus remains behind in the aqueous phase because of the relatively high solubility of the β-lactam nucleus in water. The aqueous phase that is obtained after isolation of the β-lactam nucleus but which still contains some β-lactam nucleus is often called the mother liquor (ML). The process according to the invention allows the loss of β-lactam nucleus to be limited. Preferably, the loss is at least 5% less, more preferably the loss of β-lactam nucleus is at least 10% less, the percentage loss being calculated by dividing the number of moles of β-lactam nucleus in the mother liquor by the number of moles of β-lactam compound employed in the deacylation reaction and multiplying the quotient by 100%.

International application WO 98/48039 discloses a process wherein a solution of 6-APA is obtained by extracting N-substituted penicillin from a fermentation broth to an organic solvent and subsequently extracting the N-substituted penicillin back to water and then treating the aqueous phase with a penicillin acylase, in which process 6-APA and phenylacetic acid are formed through enzymatic deacylation of the N-substituted penicillin. In this reaction, however, the pH of the aqueous phase decreases because of the phenylacetic acid that is formed. This is disadvantageous for the equilibrium of the enzymatic deacylation reaction. For that reason, in the process of WO 98/48039 the pH is maintained, with the aid of ammonia or an aqueous alkaline solution, at such a value that the equilibrium of the enzymatic deacylation reaction lies on the product side. Following the enzymatic deacylation, 6-APA and/or phenylacetic acid may be isolated from the aqueous phase.

A drawback of the known process is that phenylacetic acid is still present in the reaction mixture after the enzymatic deacylation reaction. In addition, the known processes for hydrolyzing Pen G produce solutions or suspensions of 6-APA in which inorganic salts are present. It is an object of the present invention to provide a process that does not have this drawback. It is also an object of the invention to provide a process that leads to the formation of less inorganic salts as a by-product.

It has now been found that the presence of inorganic salts has an adverse effect on the synthesis of antibiotics. The known solutions or suspensions of 6-APA that are obtained from an enzymatic deacylation reaction of Pen G without isolating and redissolving 6-APA or without application of purification techniques are therefore less suited as a starting material for enzymatic synthesis of β-lactam antibiotics.

In a preferred embodiment of the process according to the invention, the process is carried out in such a manner that no inorganic salts can form later. This may be accomplished by using as a starting material for example the free acid of Pen G and at the same time not correcting the pH during the enzymatic deacylation reaction or by using as a starting material the free acid of Pen G and correcting the pH with a base that does not lead to the formation of an inorganic salt. Accordingly, in a preferred embodiment the process according to the invention is carried out in the presence of at least a compound chosen from the group of esters or amides of phenylglycine or hydroxyphenylglycine, preferably D-(−)-parahydroxyphenylglycine methyl ester (HPGM), D-(−)phenylglycine methyl ester (PGM), D-(−)-parahydroxyphenylglycinamide (HPGA) and D-(−)-phenylglycinamide(PGA). This process also prevents the formation of inorganic salts when another β-lactam compound is used instead of PenG.

In an embodiment of the process according to the invention the β-lactam nucleus precipitates during the enzymatic deacylation reaction. Precipitation may for example be accomplished by using a high concentration of Pen G. In a preferred embodiment, a solution of Pen G in an organic solvent is contacted with water and an enzyme so that a reaction mixture is formed having a concentration of the product 6-APA and a pH, preferably in the region of the isoelectrical point of 6-APA, at which 6-APA precipitates. Precipitation of 6-APA leads to a higher degree of conversion of the deacylation reaction, because the product 6-APA is withdrawn from the solution. Precipitation of 7-ADCA may be achieved similarly to the manner described here for 6-APA.

An advantage of the process according to the invention is that the loss of β-lactam nucleus in comparison with the loss encountered in existing processes may be strongly reduced, because the nucleus need not be isolated from the aqueous phase. The process according to the invention may therefore result in a higher efficiency than existing processes. Furthermore, less inorganic salts are produced as unwanted by-products. The salts that are formed in the existing processes are a burden on the environment.

A solution or suspension of the β-lactam compound in an organic solvent and a solution or suspension containing the enzyme in water may be contacted with one another in various ways, for example in a batch process, in a co-current process or in a countercurrent process. In the context of the present application, a batch process is understood to be a process wherein the organic phase and the aqueous phase are mixed in one vessel and subsequently separated and wherein no fresh water or organic solvent is added. In the context of the present application, a co-current process is understood to be a process such as the abovementioned batch process after which the aqueous phase is mixed with and then separated from fresh organic solvent one or more times, whereby the enzyme may flow along with the aqueous phase, or after which the organic phase is mixed with fresh water and fresh enzyme.

Preferably the process according to the invention is carried out in the form of a co-current process. This has the advantage that a purer product is obtained than in a batch process. Still more preferably the process according to the invention is carried out in the form of a countercurrent process. This may be effected for example in a setup as shown in FIG. 1. In FIG. 1 the countercurrent principle is shown for a deacylation reaction with Pen G, but of course the same principle may also be applied when a different β-lactam compound is chosen. In the process mixers and settlers are used.

A mixer is understood to be a vessel in which water and the organic solvent have been or are being mixed. A settler is understood to be a vessel in which the reaction mixture has been separated into two phases, an aqueous phase and an organic phase, or in which the mixture is being separated into two phases.

The bottom layer (BL) in FIG. 1 is the aqueous phase, the upper layer (UL) is the organic phase.

m indicates the number mixers and settlers placed between the mixer where Pen G in organic solvent is introduced and the settler where the carboxylic acid (PAA) in organic solvent is removed from the counter current system.

n indicates the number of mixers and settlers in which the aqueous layer is washed with the organic solvent between the mixer where the organic solvent is introduced and the settler and mixer where Pen G in organic solvent is introduced. The more washing operations are carried out, the purer the final product will be, and the purer the final product, the higher the yield. This principle is known to those skilled in the art.

[image]

In FIG. 1, enzyme may be introduced into the system along with the water to flow along with the water and to exit the system along with 6-APA. An alternative is immobilised enzyme that is present in all mixers. In that case, a provision is needed in the mixer to keep the enzyme in the mixer, for example a filter that retains immobilised enzyme.

6-APA may flow through the system as 6-APA dissolved in the aqueous bottom layer. It is also possible for 6-APA to precipitate during the process. Precipitated 6-APA is preferably carried along by the aqueous bottom layer, which aqueous product stream is a suspension of 6-APA. This may be achieved by for example filtering the organic phase so that 6-APA is retained, or by centrifuging the reaction mixture after which the aqueous phase with precipitated 6-APA and the organic phase may be separated.

Immobilized enzyme may be carried along by the aqueous phase in the same way as precipitated 6-APA.

In case use is made of immobilised enzyme and precipitated 6 APA, one may choose to leave the enzyme in the mixers. 6-APA and enzyme may be separated using for example a sieve through which solid 6-APA can pass and immobilised enzyme cannot pass.

An advantage of the countercurrent process is that this process results in a higher efficiency of the process, which means that more β-lactam nucleus is obtained and the nucleus is of a purer quality than in a batch process. In the present context, purer means that less β-lactam compound and less carboxylic acid are present in the amount of crude β-lactam nucleus obtained.

In an embodiment of the process according to the invention wherein 6-APA crystallizes during the enzymatic deacylation, which has a favourable effect on the deacylation reaction, it is advantageous for the solid 6-APA to be carried along with the aqueous phase in a countercurrent or co-current process.

The invention also relates to aqueous solutions or suspensions of 6-APA or 7-ADCA obtainable by the process according to the invention.

The invention also relates to a process for the enzymatic preparation of a β-lactam antibiotic by reacting a β-lactam nucleus with a side chain presursor, characterised in that the β-lactam nucleus used originates from an aqueous solution or suspension according to the invention, that is, the aqueous solution or suspension is used directly, without the β-lactam nucleus having been isolated therefrom and without the solution or suspension having been subjected to a processing step.

In a preferred embodiment the enzymatic preparation of a β-lactam antibiotic is carried out using a salt-free solution or suspension.

To that end, the process comprises the following steps: an enzymatic deacylation of a β-lactam compound, which compound comprises a β-lactam nucleus with a side chain attached to it via an amide bond, and which deacylation is carried out in a mixture of water and an organic solvent and which deacylation leads to a β-lactam nucleus and a carboxylic acid, is carried out at a pH value of between 2 and 6, so that the carboxylic acid is extracted almost completely in situ to the organic solvent, whereupon the organic solvent that forms an organic phase is separated from the aqueous phase, whereupon the aqueous phase that contains the β-lactam nucleus is contacted with a side-chain precursor and an enzyme that catalyzes the coupling of the side-chain precursor and the nucleus. In this embodiment, preferably more than 90%, more preferably 95% and most preferably 99% of the carboxylic acid is extracted to the organic solvent.

The more mixers and settlers are used, the more carboxylic acid will be extracted. Extraction may also be optimized by selecting a good solvent and by using the optimum pH.

All side chain precursors known in the art may be used in the enzymatic preparation of an antibiotic. Preferably, a side-chain precursor is defined as an ester or amide of a side chain in a β-lactam antibiotic. More preferably, the side-chain precursor used is an ester or amide of D-phenylglycine or D-p-hydroxyphenylglycine or, most preferably, a methyl ester, ethyl ester, n-propyl ester or a hydroxyethyl ester.

The side-chain precursor may be coupled to the nucleus using any enzyme known for this reaction, such as penicillin acylases from class E.C.3.5.1.11 or α-amino acid ester hydrolases from class E.C.3.1.1.43. Such enzymes are described in for example WO98/48038. Antibiotics that may be prepared with the process according to the invention are for example ampicillin, amoxicillin, cefadroxil, cephalexin, cefradin, cefprozil and cefaclor.

### EXAMPLES

Abbreviations

| | |
|---|---|
| 6-APA | 6-aminopenicillanic acid |
| Pen G | Penicillin G |
| 7-ADCA | 7-aminodesacetoxycefalosporanic acid |
| MTBE | methyl-t-butyl ether |
| HPGM | D-(−)-parahydroxyphenylglycine methyl ester |
| PGM | D-(−)-phenylglycine methyl ester |
| HPGA | D-(−)-parahydroxyphenylglycinamide |
| PGA | D-(−)-phenylglycinamide |
| pen acylase | penicillin acylase |

Raw Materials

Pen acylase was obtained from Escherichia Coli, ATCC 11105, as described in international patent application WO97/04086.

Immobilised Pen acylase was obtained as described in European patent application EP-A-0 222 462. Gelatin and chitosan were used as gelating agents. Glutaraldehyde was used as crosslinking agent.

Method of Determining IU (International Units)

1 Unit is the activity needed to convert 1 micromole of Pen G in one minute under standard conditions. The standard conditions are a pH value equal to 8, a temperature of 28° C., a 10% solution of the potassium salt of Pen G in water (% by mass), 50 mM potassium phosphate buffer, titrimetric method using NaOH as titrant.

### Example 1

3.7 g (10 mmol) of the potassium salt of Pen G was dissolved in a mixture of 100 ml of water and 100 ml of methyl-t-butyl ether. The mixture was adjusted to pH 2.6 at room temperature with the aid of 6 M sulphuric acid. The layers were separated. 100 ml of water was added to the organic layer (95 ml, HPLC analysis indicated that this solution contained 9.7 mmol Pen G). To the mixture was added 1.36 g (7.5 mmol) of D-p-hydroxyphenylglycine methyl ester (HPGM). At that point the pH of the aqueous layer was approximately 4.5. Subsequently, immobilised pen acylase enzyme with 4200 Units of activity was added. The mixture was stirred for 3 hours at room temperature. After stirring, the pH of the aqueous layer was approximately 4.8. Subsequently, the concentrations of Pen G, phenylacetic acid, 6-APA and HPGM in both layers were determined through HPLC. The result of HPLC analysis is shown in Table 1.

TABLE 1

| | | | [phenyl acetic | | | | Phenylacetic | | |
|---|---|---|---|---|---|---|---|---|---|
| | volume | [Pen G] | acid] | [6-APA] | [HPGM] | Pen G | acid | 6-APA | HPGM |
| layer | (ml) | (mM) | (mM) | (mM) | (mMm) | (mmol) | (mmol) | (mmol) | (mmol) |
| water | 115 | 23.1 | 14.0 | 42.1 | 52.5 | 2.7 | 1.6 | 4.8 | 6.0 |
| MTBE | 65 | 6.2 | 68.4 | 0.0 | 0.0 | 0.4 | 4.4 | 0.0 | 0.0 |

4.8 mmol 6-APA corresponds to 48% efficiency relative to the amount (in moles) of potassium salt of Pen G used.
MTBE stands for methyl-t-butyl ether.

### Example 2

1.86 g (5 mmol) of potassium salt of Pen G was dissolved in a mixture of 20 ml of water and 20 ml of n-butyl acetate. The mixture was brought to pH=2.6 at room temperature with the aid of 6 M sulphuric acid. The layers were separated. To the organic layer was added 30 ml of n-butyl acetate and 50 ml of water. To the mixture was added 0.36 g (2.0 mmol) of HPGM. Subsequently immobilised pen acylase enzyme with 2100 Units of activity was added. After stirring for 1 hour, the pH of the aqueous layer was approximately 4.4. The mixture was stirred for 5½ hours at room temperature. Subsequently, the concentrations Pen G, phenylacetic acid, 6-APA and HPGM in both layers were determined through HPLC. The result is shown in Table 2.

TABLE 2

| | | [phenylacetic | | |
|---|---|---|---|---|
| | [Pen G] | acid] | [6-APA] | [HPGM] |
| Layer | (mM) | (mM) | (mM) | (mM) |
| Water | 16.1 | 4.5 | 49.5 | 37.5 |
| n-butyl acetate | 13.7 | 43.6 | N.d. | 0.0 |

n.d.: Not determined

49.5 mM of 6-APA in 50 ml of water corresponds to 50% yield relative to the potassium salt of Pen G (in moles) used.

### Example 3

9.3 g of potassium salt of Pen G (25 mmol) was dissolved in a mixture of 250 ml of water and 250 ml of n-butyl acetate. The mixture was brought to pH 2.6 at room temperature with the aid of 6 M sulphuric acid. The layers were separated. 50 ml of water and immobilised pen acylase enzyme with 10500 Units of activity was added to the organic layer. The mixture was stirred for 16 hours at room temperature, in which period the pH rose from pH 2.7 to pH 3.5 and a white precipitate had formed. The reaction mixture was filtered with a sieve with a pore size of 100 μm; the immobilised enzyme did not pass through the sieve and the white product in water/n-butyl acetate did pass. The filtrate was transferred into a separating funnel and the layers were separated. The bottom layer was filtered through a glass filter, the white product being retained on the filter. The clear filtrate was added to the immobilised enzyme on the sieve, which contained traces of white product, the filtrate of the sieve was filtered again through the glass filter, and the procedure was repeated until the white product and the immobilised enzyme were separated. Finally, the upper layer was also filtered through the glass filter. The isolated product, i.e. the product on the filter, was dried and analysed. The product (4.14 g) contained 97.4% 6-APA, corresponding to 18.6 mmol of 6-APA. Efficiency relative to the potassium salt of Pen G in moles used: 75%. The aqueous layer contained 4 mmol (2%) of 6-APA.

### Example 4

14 g (37.5 mmol) of potassium salt of Pen G was dissolved in a mixture of 250 ml of water and 250 ml of methyl-t-butyl ether. The mixture was brought to pH 2.6 at room temperature with the aid of 6 M sulphuric acid. The layers were separated. Analysis by HPLC indicated that the organic layer (238 ml) contained 35.2 mmol of Pen G. The aqueous layer contained 0.7 mmol of Pen G.

250 ml of water was added to the organic layer. 0.91 g (5.0 mmol) of HPGM was added to the mixture. Subsequently, immobilised pen acylase enzyme with 13000 Units of activity was added. The pH of the aqueous layer then was approximately 3.7. The mixture was stirred at room temperature. After 50 minutes a white precipitate developed. The mixture was stirred at room temperature for 5 hours and 40 minutes in total. The concentrations of Pen G, phenylacetic acid, 6-APA and HPGM in both layers were determined at regular intervals by HPLC, as was the pH of the aqueous layer (see Table 3). The samples of both layers were filtered for analysis, the concentrations of the dissolved components mentioned were determined.

TABLE 3

| | | | | [phenylacetic | | |
|---|---|---|---|---|---|---|
| time | | | [Pen G] | acid] | [6-APA] | [HPGM] |
| (min) | pH | phase | (mM) | (mM) | (mM) | (mM) |
| 8 | 3.72 | water | 22.8 | 1.3 | 31.9 | 19.3 |
| | | MTBE | 93.7 | 38.2 | 0.0 | 0.0 |
| 30 | 3.81 | water | 18.3 | 2.9 | 61.5 | 18.2 |
| | | MTBE | 57.4 | 76.5 | 1.9 | 0.0 |
| 60 | 3.90 | water | 16.4 | 3.8 | 49.0 | 18.2 |
| | | MTBE | 41.6 | 94.9 | 0.0 | 0.0 |
| 120 | 4.00 | water | 14.0 | 5.0 | 37.4 | 18.8 |
| | | MTBE | 26.6 | 113.2 | 0.0 | 0.0 |
| 331 | 4.15 | water | 11.5 | 6.3 | 31.0 | 19.3 |
| | | MTBE | 16.4 | 128.7 | 0.0 | 0.0 |

After 5 hours and 40 minutes the reaction mixture was filtered through a sieve with a pore size of 100 μm. The immobilised enzyme did not pass the sieve, the solid, white product, together with organic and aqueous phase, did. The mixture without immobilised enzyme was transferred into a separating funnel, and the bottom layer was drained and filtered through a glass filter, the white product being retained on the filter. The filtrate was added to the immobilised enzyme (containing traces of solid product), and filtering was effected first through the sieve and then through the glass filter. The procedure was repeated a number of times so that white product in the presence of the immobilised enzyme was flushed to the glass filter. Eventually, the top layer from the separating funnel was also filtered through the glass filter. The layers in the filtrate were separated. The solid, white product was dried. There were 5 product streams:

aqueous layer after extraction of Pen G to methyl-tertiary butyl ether (MTBE) (containing 0.7 mmol of Pen G)

immobilised enzyme with adhering reaction mixture

filtered aqueous layer, 210 ml

filtered MTBE layer, 85 ml (a proportion of the MTBE evaporated during filtration)

dried product, 4.4 g (content of 6-APA 95.8%, content of Pen G 1.9%)

The immobilised enzyme with adhering reaction mixture was stirred in 400 ml of water, the pH was brought to pH 7 with 1 M NaOH solution and stirred at room temperature. The mixture was sampled and analysed to determine the losses. The other process streams were also analysed. The results are stated in Table 4.

TABLE 4

| | Pen G | | 6-APA | HPGM |
|---|---|---|---|---|
| Process stream | (mmol) | PAA (mmol) | (mmol) | (mmol) |
| aqueous layer after | 0.7 | | | |
| extraction | | | | |
| immobilised enzyme | 0.1 | 4.3 | 5.1 | 0.0 |
| (after diluting and | | | | |
| adjusting pH to pH = 7) | | | | |
| filtered aqueous layer | 2.7 | 3.2 | 5.4 | 3.8 |
| filtered MTBE layer | 2.4 | 20.1 | 0.0 | 0.0 |
| isolated product | 0.2 | 0.0 | 19.5 | 0.0 |

The percentage of 6-APA in the filtered aqueous layer+isolated product was 66% ([5.4+19.5]/37.5) relative to the potassium salt of Pen G (in moles) used.

### Example 5

14 g (37.5 mmol) of the potassium salt of Pen G was dissolved in a mixture of 250 ml of water and 250 ml of methyl-t-butyl ether. The mixture was brought to pH 2.6 with 6 M sulphuric acid at room temperature. The layers were separated. HPLC indicated that the organic layer (238 ml) contained 35.2 mmol of Pen G. The aqueous layer contained 0.7 mmol of Pen G.

250 ml of water was added to the organic layer. 0.91 g (5.0 mmol) of HPGM was added to the mixture. Subsequently, immobilised pen acylase enzyme with 13000 Units of activity was added. The pH of the aqueous layer then was approximately 3.7. The mixture was stirred at room temperature. After 50 minutes a white precipitate developed. The mixture was stirred at room temperature for 2 hours and 10 minutes in total. The concentrations of Pen G, phenylacetic acid, 6-APA and HPGM in both layers were determined by HPLC, as was the pH of the aqueous layer (see Table 5). The samples of both layers were filtered for analysis, the concentrations of the dissolved components mentioned were determined.

TABLE 5

| | | | | [phenylacetic | | |
|---|---|---|---|---|---|---|
| Time | | | [Pen G] | acid] | [6-APA] | [HPGM] |
| (min) | pH | Phase | (mM) | (mM) | (mM) | (mM) |
| 132 | 4.05 | water | 14.0 | 5.1 | 39.3 | 18.8 |
| | | MTBE | 24.4 | 122.1 | 0.0 | 0.0 |

Subsequently the mixture was transferred to a separating funnel and the layers were separated into an organic layer containing solid, white product and a little immobilised enzyme and an aqueous layer containing solid, white product and the greater part of the immobilised enzyme.

250 ml of MTBE was added to the aqueous layer and the mixture was stirred at room temperature. 250 ml of water and immobilised enzyme with 4000 Units of activity were added to the organic layer and the mixture was stirred at room temperature. The concentrations of Pen G, phenylacetic acid, 6-APA and HPGM in both layers of both mixtures were determined by HPLC, as was the pH of the aqueous layers (see Table 6). The samples of both layers were filtered for analysis, the concentrations of the disssolved components mentioned were determined.

| | time after | | | | | | |
|---|---|---|---|---|---|---|---|
| | adding fresh | | | [Pen G] | [phenylacetic | | |
| mixture | phase (min) | pH | phase | (mM) | acid] (mM) | [6-APA] (mM) | [HPGM] (mM) |
| aqueous | 8 | 4.50 | water | 7.5 | 0.6 | 34.7 | 18.8 |
| layer + fresh | | | MTBE | 4.3 | 8.1 | 0.0 | 0.0 |
| MTBE | 192 | 4.94 | water | 3.2 | 2.2 | 24.5 | 18.8 |
| | | | MTBE | 0.6 | 15.4 | 0.0 | 0.0 |
| MTBE layer + fresh | 14 | 3.86 | water | 3.8 | 5.1 | 16.2 | 0.0 |
| water and | | | MTBE | 14.2 | 136.8 | 0.0 | 0.0 |
| Enzyme | 156 | 3.90 | water | 3.5 | 5.9 | 16.6 | 0.0 |
| | | | MTBE | 10.5 | 146.3 | 0.0 | 0.0 |

The results in Table 6 show that mixing the aqueous layer, containing immobilised enzyme, with fresh MTBE caused the conversion of Pen G to 6-APA and phenylacetic acid to progress further. Mixing of the MTBE layer with fresh water+immobilised enzyme ensured that also the conversion of Pen G to 6-APA and phenylacetic acid progressed further. This given, in combination with a countercurrent principle as shown in FIG. 1, ensures that the conversion of Pen G to 6-APA and FA will be almost complete providing the number of countercurrent steps is sufficient.

Preparation of Alpha Amino Acid Ester Hydrolase-Enzyme

The organism Acetobacter pasteurianus (ATCC6033) was cultured as described in T. Takeshi ao., J. Am. Chem. Soc., 94, 4035 (1972). The cells were harvested by filtration with a Membralox 20 nm membrane. The retentate was homogenized with an MC-4 APV Gaulin homogeniser at 600 bar. 10% dicalite 4108 was added to the mixture and cell residues were removed by filtration with a Schule filter press (KuKME 800/VI So VE(EX)-2). The filtrate was concentrated using a 50 kD DDS membrane. Ammonium sulfate was added to the cell-free extract so obtained to a concentration of 243 g of ammonium sulfate per litre. The mixture was mixed with a hydrophobic resin (Phenyl Sepharose) and stirred overnight. The mixture was poured into a column and the first fraction was discarded. Elution was carried out using 194, 146, 97, 49 and 0 g of ammonium sulfate/litre of solution. Ammonium sulfate was added to the eluates until the ammonium sulfate concentration was 243 g/l. The formed precipitate was centrifuged and the pellet was washed with 20 mM tris buffer (pH=7) containing 243 g of ammonium sulfate/litre. The pellet was dissolved in 20 mM phosphate buffer (pH=6.0) containing 0.5 g/l of Bovine Serum Albumin (BSA). Further purification was carried out by cation exchanger chromatography (SP Sepharose). The dissolved pellet was diluted and transferred to the cation exchanger. Elution was carried out with a linear gradient (start at 100% 20 mM phosphate buffer+0.5 g/l of BSA, end at 80% 20 mM phosphate buffer+0.5 g/l of BSA and 20% 20 mM phosphate buffer+0.5 g/l of BSA containing 1 M NaCl). The eluate was collected in fractions and tested for activity as follows: To a portion of a fraction was added solid 6-APA and solid HPGM (concentration in mixture [6-APA]=[HPGM]=50 mM). The pH of the mixture was kept at pH=6.0-6.4 by adding solid HPGM. HPLC revealed the presence of amoxicillin in the (%-amino acid ester hydrolase-containing fractions. The a-amino acid ester hydrolase-containing fractions can be rendered salt-free by means of dialysis (Pierce slide analyzer dialysis membrane, 10000 MWCO).

### Example 7

A solution was prepared from 1.08 g of 6-APA, 0.91 g of HPGM in 50 ml of water. To 0.25 ml of this solution was added 0.2 ml of the enzyme solution obtained as described in Example 1 (rendered salt-free by dialysis) as well as 0.05 ml of one of the solutions described below. The mixture was mixed at 20° C. and the pH was measured. During the reaction, solid HPGM was added so that the pH remained between 6.0 and 6.4. Samples were taken during the reactions and analyzed for 6-APA, HPGM, amoxicillin and HPG.

The following solutions were prepared:

0.20 g of NaCl in 6.8 ml of water

0.9 of (NH₄)₂ SO₄ in 3.1 ml of water

0.17 g of FA in 5 ml of water. The pH was adjusted to 6.2 with ammonia.

0.73 g of the potassium salt of Pen G in 8.5 ml of water

The concentrations of reactants and products after 60 minutes were as follows for each reaction (see Table 7).

TABLE 7

| | [6-APA] | [HPGM] | [amoxicillin] | [HPG] | | [amoxicillin] + [HPG] |
|---|---|---|---|---|---|---|
| Addition | (mM) | (mM) | (mM) | (mM) | [amoxicillin]/[HPG] | (mM) |
| 0.05 ml of water | 26.0 | 27.6 | 24.2 | 14.5 | 1.7 | 38.7 |
| 0.05 ml of NaCl solution | 32.4 | 34.9 | 20.6 | 18.8 | 1.1 | 39.4 |
| 0.05 ml of (NH₄)₂SO₄ solution | 40.8 | 44.4 | 12.3 | 21.1 | 0.6 | 33.4 |
| 0.005 ml of FA solution and 0.045 ml | 25.9 | 28.3 | 21.1 | 15.7 | 1.3 | 36.8 |
| of water | | | | | | |
| 0.01 ml of FA solution and 0.04 ml of | 26.2 | 32.8 | 21.4 | 17.0 | 1.3 | 38.4 |
| water | | | | | | |
| 0.05 ml of FA solution | 36.3 | 38.5 | 14.4 | 19.0 | 0.8 | 33.4 |
| 0.01 ml of Pen G P solution and 0.04 ml | 25.3 | 27.0 | 22.8 | 18.2 | 1.3 | 41.0 |
| of water | | | | | | |
| 0.05 ml of Pen G P solution | 38.8 | 31.9 | 15.6 | 19.1 | 0.8 | 34.7 |

1. Process for the preparation of an aqueous solution or suspension of a β-lactam nucleus wherein an enzymatic deacylation of a β-lactam compound, which compound comprises a β-lactam nucleus with a side chain coupled to it via an amide bond, and which deacylaton reaction is carried out in a mixture of water and an organic solvent, which organic solvent and water are able to form two phases, and which deacylation leads to a β-lactam nucleus and a carboxylic acid, is carried out at a pH value of between 2 and 6.

2. Process according to claim 1, characterised in that the β-lactam compound is penicillin G.

3. Process according to claim 1 or claim 2, comprising the steps of contacting a solution containing a free add of a β-lactam compound in an organic solvent with a solution or suspension of a penicillin acylase in water at a pH value in the range from 2 to 6, and resulting in an organic phase rich in carboxylic acid and an aqueous phase rich in the β-lactam nucleus, which phases may be recovered separately.

4. Process according to any one of claims 1-3, characterised in that the enzymatic deacylation is carried out at a pH value of between 3.5 and 5.

5. Process according to any one of claims 1-4, characterised in that HPGM, HPGA, POM or PGA is present during the enzymatic deacylation.

6. Process according to any one of claims 1-5, characterised in that butyl acetate or methyl-tertiary-butyl ether is used as organic solvent.

7. Process according to any one of claims 1-6, characterised in that the enzymatic deacylation is carried out with the countercurrent principle being applied for the organic phase and the aqueous phase.

8. Process according to any one of claim 1-7, characterised in that the process is carried out so that 6-APA crystallizes during the enzymatic deacylation.

9. Process according to any one of claims 1-8, characterised in that the enzyme is immobilised.

10. Process according to any one of claims 1-9, characterised in that no inorganic acid or inorganic base is added during the process.

11. Aqueous solution or suspension of the β-lactam nucleus obtainable by the process according to any one of claims 10.

12. Process for the enzymatic preparation of an antibiotic by reacting a β-lactam nucleus with a side-chain precursor, characterised in that a solution or suspension according to claim 11 is used.

13. Process for the enzymatic preparation of an antibiotic comprising:
producing a β-lactam nucleus and a carboxylic acid by an enzymatic deacylaction reaction of a β-lactam compound, wherein said β-lactam nucleus and said carboxylic acid are distributed in different amounts over an aqueous phase and an organic phase during the enzymatic deacylation; and
reacting said β-lactam nucleus with a side-chain precursor.

14. Process for the enzymatic preparation of an antibiotic wherein an enzymatic deacylation of a β-lactam compound, which compound comprises a β-lactam nucleus with a side chain coupled to it via an amide bond and which deacylation is carried out in a mixture of water and an organic solvent and which deacylation leads to a β-lactam nucleus and a carboxylic acid, is carried out at a pH value of between 2 and 6 so that the carboxylic acid is extracted in situ to the organic solvent, whereupon the aqueous phase containing the β-lactam nucleus is contacted with a side-chain precursor and an enzyme that catalyzes the coupling of the side-chain precursor and the β-lactam nucleus.

15. Process for the enzymatic preparation of an antibiotic wherein an enzymatic deacylation of a β-lactam compound, which compound comprises a lactam nucleus with a side chain coupled to it via an amide bond and which deacylation is carried out in a mixture of water and an organic solvent and which deacylation leads to a β-lactam nucleus and a carboxylic acid, is carried out at a pH value of between 2 and 6 so that 95%, more preferably 99% of the carboxylic acid is extracted in situ to the organic solvent, whereupon the aqueous phase containing the β-lactam nucleus is contacted with a side-chain precursor and an enzyme that catalyzes the coupling of the side-chain precursor and the β-lactam nucleus.
